(19) United States
(12) Patent Application Publication
(10) Pub. No.: US 2026/0207806 A1
(43) Pub. Date: Jul. 23, 2026

(54) PORTABLE OZONE DISINFECTION DEVICE

(71) Applicant: DCSTAR INC, New York, NY (US)

(72) Inventors: David LUO, New York, NY (US); Ligui HE, New York, NY (US)

(21) Appl. No.: 19/027,721

(22) Filed: Jan. 17, 2025

(51) Int. Cl. A61L 2/20

(52) U.S. Cl. CPC A61L 2/202; A61L 2103/15; A61L 2202/122; A61L 2202/15; A61L 2202/16

(57) ABSTRACT

A portable ozone disinfection device configured for disinfecting and sterilizing respiratory-related devices or respiratory accessories. Specifically, provided is an ozone disinfection device that enhances usability and comfort. The device includes a functional component end enclosed by at least one housing and an external disinfection space. The functional component end includes components such as an ozone generation system, an ozone recovery system, and an ozone operation system, the ozone operation system having at least one switch. The housing also includes at least one interface configured to connect with a hose for the delivery of ozone gas. This interface has various forms.

### TECHNICAL FIELD

The present disclosure relates to an ozone disinfection device configured to disinfect respiratory-related devices and respiratory accessories. The device is configured to distribute ozone into the pipelines connected to respiratory-related devices and/or respiratory accessories and output ozone for cleaning and disinfection.

### BACKGROUND

Obstructive Sleep Apnea (OSA) is a common sleep-related breathing disorder and constitutes a significant proportion of respiratory-related illnesses. Statistical analyses from relevant studies, based on the diagnostic criteria of the American Academy of Sleep Medicine (AASM), indicate that approximately 936 million adults aged 30 to 69 worldwide suffer from mild to severe OSA. Among them, around 425 million adults aged 30 to 69 are diagnosed with moderate to severe OSA. In the United States, approximately 26% of adults aged 30 to 70 are affected by OSA, characterized by five or more apnea or hypopnea events per hour. This condition affects approximately 34% of males and 17% of females in the same age group. Moreover, studies estimate that globally, approximately 936 million adults experience mild to severe OSA, with about 425 million suffering from moderate to severe OSA.

There are various treatments for Obstructive Sleep Apnea (OSA), including lifestyle changes, oral appliance therapy, continuous positive airway pressure (CPAP) therapy, and various surgical interventions. Among these, CPAP therapy is the most commonly chosen treatment by users. This method involves CPAP devices, breathing tubes, headgear, and user interface cushions, such as masks, nasal masks, or oronasal masks. CPAP devices require prolonged and regular use, often worn continuously during sleep, making the cleanliness of the respiratory equipment and accessories crucial for users.

Respiratory-related devices and respiratory accessories come into prolonged contact with the user's respiratory tract. The suitable temperature and humidity in these devices can create a breeding ground for bacteria, viruses, fungi, and other pathogens. If the equipment and accessories are not regularly disinfected, these microorganisms can enter the body through the user's respiratory tract, leading to respiratory diseases or more severe infections. This risk is particularly heightened for users with weaker immune systems. Therefore, all components of respiratory-related devices (such as masks, tubing, and water tanks) should be cleaned regularly. Selecting appropriate disinfection methods and materials for respiratory-related devices is critical for maintaining the user's health. Improper disinfection can lead to respiratory diseases such as pneumonia or sinusitis and exacerbate issues due to the accumulation of bacteria or fungi. Moreover, incorrect cleaning or disinfection methods may cause aging or damage to equipment components, reducing their lifespan. The buildup of dirt can also obstruct the normal flow of air, compromising the effectiveness of the respiratory device and diminishing its therapeutic benefits for users with sleep-disordered breathing. Thus, regular and proper cleaning and disinfection of respiratory devices are essential to ensure the hygiene, functionality, and health safety of users.

Ozone, as one of the most effective oxidizing agents and disinfectants available on the market, can typically reduce microbial populations by 5-6 log levels within 2-3 minutes. Its application is safe and controllable, making it an ideal method for disinfecting respiratory-related equipment and respiratory accessories. Ozone technology represents an innovative disinfection method capable of effectively eliminating bacteria and viruses. Ozone disinfectors have become the preferred choice for some CPAP device users as they can penetrate device tubing to achieve comprehensive disinfection and pathogen elimination.

### SUMMARY

This disclosure addresses the aforementioned challenges by providing an ozone disinfection device that is user-friendly, reduces user costs, and ensures comfortable usage.

In one embodiment, a portable ozone disinfection device is provided, configured to disinfect and sterilize respiratory-related equipment or respiratory accessories. The portable ozone disinfection device includes an ozone generation system configured to produce and output ozone gas; an ozone recovery system including at least one pump or fan, the at least one pump or fan being configured to suction the ozone gas; and an external disinfection space, configured to be fluidly connectable to the ozone generation system and to accommodate the respiratory accessories. The ozone gas is generated and flows out from the ozone generation system, passes through the external disinfection space, and is subsequently recovered by the ozone recovery system. The ozone disinfection device further includes at least one interface, the at least one interface being configured to fluidly connect to the external disinfection space, in which the external disinfection space is configured to be sealable and gas-impermeable and to accommodate a minimum ozone volume of 0.125 liters.

In one embodiment, the ozone disinfection device further includes an ozone operation system configured to control the operation of the ozone disinfection device, and the ozone operation system includes at least one switch.

In one embodiment, the ozone recovery system further includes at least one recovery device.

In one embodiment, the ozone recovery system is configured to connect to the external disinfection space and perform recovery and filtration.

In one embodiment, the ozone recovery system is configured to connect with the ozone generation system and perform recovery and filtration.

In another embodiment, a portable ozone disinfection device is provided, configured to disinfect and sterilize respiratory-related equipment or respiratory accessories. The portable ozone disinfection device includes an ozone generation system configured to produce and output ozone gas; an ozone recovery system including at least one pump or fan, the at least one pump or fan being configured to suction gas, in which the ozone recovery system further includes at least one recovery device; and an external disinfection space configured to be fluidly connectable to the ozone generation system and configured to be sealable, gas-impermeable, and to accommodate the respiratory accessories. The ozone disinfection device further includes at least one interface, the at least one interface being configured to fluidly connect to the external disinfection space, in which, during operation, a volume of the ozone gas generated by the ozone generation system is at least one-third of a volume of the external disinfection space.

In one embodiment, the ozone disinfection device further includes an ozone operation system configured to control the operation of the ozone disinfection device, and the ozone operation system includes at least one switch.

In one embodiment, the external disinfection space includes a flexible material.

In one embodiment, the external disinfection space includes a rigid material.

In one embodiment, the ozone generation system is configured to be separated from the external disinfection space.

In one embodiment, the external disinfection space includes at least one opening configured to switch between an open state and a closed state.

In yet another embodiment, a portable ozone disinfection device is provided, configured to disinfect and sterilize respiratory-related equipment or respiratory accessories. The portable ozone disinfection device includes an ozone generation system configured to produce and output ozone gas; an ozone recovery system including at least one pump or fan, the at least one pump or fan being configured to suction gas; and an external disinfection space configured to be fluidly connectable to the ozone generation system and configured to be sealable, gas-impermeable, and to accommodate the respiratory accessories. The ozone gas is generated and flows out from the ozone generation system, passes through the external disinfection space, and is subsequently recovered by the ozone recovery system. The ozone disinfection device further includes at least one interface, the at least one interface being configured to fluidly connect to the external disinfection space, in which a volume of the gas suctioned by the at least one pump or fan during operation is greater than or equal to a volume of the external disinfection space.

In one embodiment, the ozone disinfection device further includes an ozone operation system configured to control the operation of the ozone disinfection device, and the ozone operation system includes at least one switch.

In one embodiment, the ozone recovery system is configured to be downstream of the ozone generation system.

In one embodiment, the ozone recovery system further includes at least one recovery device.

In one embodiment, the ozone disinfection device further includes at least one gas outlet, and the at least one gas outlet is provided downstream of the external disinfection space.

In another embodiment, a portable ozone disinfection device is provided, configured to disinfect and sterilize respiratory-related equipment or respiratory accessories. The portable ozone disinfection device includes an ozone generation system configured to produce and output ozone gas; an ozone recovery system including at least one pump or fan, the at least one pump or fan being configured to suction gas; and an external disinfection space configured to be fluidly connectable to the ozone generation system and configured to be sealable, gas-impermeable, and to accommodate the respiratory accessories. The ozone gas is generated and flows out from the ozone generation system, passes through the external disinfection space, and is subsequently recovered by the ozone recovery system. The ozone disinfection device further includes at least one interface, the at least one interface being configured to fluidly connect to the external disinfection space, in which, during operation, a volume of the ozone gas generated by the ozone generation system is at least one-third of a volume of the external disinfection space. The ozone disinfection device further includes an ozone operation system configured to control the operation of the ozone disinfection device, and the ozone operation system includes at least one switch.

In one embodiment, the ozone recovery system further includes at least one recovery device.

In one embodiment, the external disinfection space includes a collection of multi-layered materials.

In one embodiment, the external disinfection space includes a single-layer material. In one embodiment, the ozone disinfection device further includes at least one gas outlet, in which the at least one gas outlet is provided downstream of the external disinfection space.

The ozone disinfection device of this disclosure offers at least the following beneficial effects:
1) This disclosure provides not only a portable ozone disinfection device for convenient use but also adds a recyclable function compared to existing ozone disinfection devices on the market. This enhancement improves the device's safety, efficiency, and reliability while maintaining its convenience. The portable ozone disinfection device is lightweight and easy to carry, offering significant convenience to users. Unlike integrated ozone disinfection devices currently available, this disclosure is more suited for use in various scenarios, such as during travel or in outdoor environments, thereby enhancing product adaptability and making it less dependent on environmental conditions. This mobility also reduces reliance on specific storage and transportation conditions, lowering costs associated with these processes. Furthermore, compared to existing products on the market, the ozone disinfection device of this disclosure includes a recyclable function that effectively processes ozone gas after use, reducing potential harm to the environment or users. The addition of an ozone recovery system makes this portable ozone disinfection device environmentally beneficial compared to other portable ozone disinfection devices with similar functionalities. It aligns with the requirements of sustainable development. The ozone recovery system ensures that after disinfection, the ozone gas is effectively converted into oxygen, preventing excessive ozone release into the environment. In traditional disinfection processes, ozone gas is often directly released after disinfection, leading to resource waste and increased energy consumption during use. By incorporating the ozone recovery system, this device maximizes ozone gas recovery, conserving energy and reducing operational pollution. The ozone recovery system in this disclosure complies with relevant environmental regulations, reducing ozone gas leakage, helping users meet environmental standards, and preventing environmental pollution caused by improper use of the device. In the current era of increased environmental awareness, this eco-friendly design enhances the social responsibility of both users and manufacturers. In summary, the portable ozone disinfection device offers high mobility and adaptability, providing users with an efficient and flexible disinfection experience. It broadens the device's applicability while meeting user demands for a green, efficient, and convenient disinfection device, delivering significant social value.
2) Additionally, the portable ozone disinfection device with recyclable functionality extends the device's lifespan and reduces replacement costs. Ozone gas is highly oxidizing, and prolonged contact with the internal materials of the device can lead to corrosion or aging of key components, resulting in performance degradation. Due to its relatively simple structure and compact design, the portable ozone disinfection device lacks excess complexity. If the internal gas pathways are not fully sealed, there is a high risk of damage to sensors or electronic components, particularly those sensitive to oxidative reactions. This disclosure addresses these challenges by using corrosion-resistant materials for constructing the housing of the ozone disinfection device, while simultaneously utilizing the ozone recovery system to minimize ozone gas accumulation inside the device and reduce the storage time of ozone gas within the device. This system prevents gas backflow or dispersal, which is crucial for protecting internal materials and extending the device's lifespan. Moreover, the external disinfection space can be tailored to user requirements by choosing different suitable materials, offering significant flexibility. This broad range of material options not only allows manufacturers to potentially lower material costs and diversify production choices but also enables the external disinfection space to have various characteristics depending on the selected materials. These characteristics might include heat resistance, moisture resistance, UV resistance, corrosion resistance, or lightweight properties, enhancing manufacturing flexibility and, more importantly, the adaptability and practicality of the product. Manufacturers can quickly adjust their designs based on market feedback, ensuring the product consistently aligns with market demands and improves user experience. Furthermore, the customizable materials and optimized design of the external disinfection space can effectively extend the device's lifespan and reduce replacement costs caused by material degradation or functional failure.
3) The portable ozone disinfection device in this disclosure, including an ozone recovery system 3, enhances the user experience when using the device for disinfection. Without the ozone recovery system to automatically reclaim ozone gas during the disinfection process, users would need to manually collect and process the ozone gas or release it directly into the environment. Firstly, high concentrations of ozone gas escaping into the environment could pose risks to users, bystanders, and the surrounding environment. When manually collecting and processing ozone gas, it is time-consuming and still carries the risk of gas leakage during handling. By incorporating the ozone recovery system into the portable ozone disinfection device, this disclosure simplifies the ozone recovery process, thereby improving the device's convenience and efficiency. In terms of portability, this disclosure uses a portable external disinfection space instead of the larger, heavier integrated external disinfection spaces found in traditional devices. The external disinfection space can feature various design forms and accommodate multiple configurations without being constrained by the functional components or housing of the ozone disinfection device. For example, in one implementation, the external disinfection space is made of flexible materials such as fabric, allowing it to be folded when not in use. This design increases the capacity of the external disinfection space for accommodating respiratory accessories during use while reducing the overall size of the ozone disinfection device when not in use, thereby significantly improving portability. This flexibility not only makes the ozone disinfection device easier to carry daily but also enhances its practicality by meeting diverse disinfection needs. It allows users to disinfect larger or multiple pieces of respiratory equipment and accessories, thereby improving disinfection efficiency.
4) The external disinfection space makes the ozone disinfection device more compact, structurally optimized, and cost-efficient. Furthermore, when combined with the advantages of the ozone recovery system, the overall cost of the ozone disinfection device is further reduced. This disclosure centralizes the design of the functional components while independently designing the external disinfection space, which is fluidly connectable to the functional components. This approach not only reduces the need for complex internal wiring and component space but also lowers the requirements for internal space and disinfection space integration. As a result, the size of the housing and the amount of material required are significantly reduced. These optimizations make storage and transportation of the ozone disinfection device more convenient and cost-effective. Cost savings are further reflected in the independent and integrated design of the functional components, which simplifies maintenance and replacement, reducing the complexity of disassembly and reassembly. Since the external disinfection space is a separate unit, the sealing between the two spaces does not require intricate sealing structure designs or multiple testing phases. In production, this disclosure simplifies assembly, further reducing labor and time costs. Moreover, the portable ozone disinfection device offers more user-friendly operation compared to integrated ozone disinfection devices. Its functional simplicity makes it easier for users to understand and operate, reducing the learning curve. This is particularly valuable for users who need frequent disinfection or for elderly users, as it provides a more accessible and convenient experience. In one embodiment, the ozone recovery system is configured to be replaceable as a standalone component. This independent configuration further enhances the usability of the ozone disinfection device by reducing the frequency and complexity of user maintenance. Users only need to periodically check the usability of the individual components of the ozone recovery system and replace it independently when necessary. This modular approach minimizes maintenance efforts while ensuring long-term functionality.
5) The ozone disinfection device in this disclosure provides users with a modular usage option, designed with a full consideration of diverse user needs and future upgrade potential. The external disinfection space allows for flexibility, enabling users to add, reduce, or replace the external disinfection space based on their needs without affecting functionality. For users requiring larger external disinfection spaces, they can purchase additional or larger external disinfection spaces individually, or increase the number of external disinfection spaces, thereby catering to a wide range of user requirements. This modular design also significantly facilitates product iteration and upgrades. For developers, upgrading the product becomes easier, as improvements to the functional component end only require adjustments and upgrades to core module components without altering the structure or internal circuitry. Moreover, such changes do not necessitate adjustments to the external disinfection space. Similarly, if the external disinfection space needs an upgrade, only its materials, size, or other features need to be redeveloped and tested, with no impact on the functional component end's structure or components. This modular approach to research and development shortens the development cycle and reduces overall development costs. Additionally, for manufacturers, the functional component end of the portable ozone disinfection device 1 and the external disinfection space can be produced independently, without the need for additional assembly. This simplifies the production process, reduces assembly complexity, and enhances production efficiency. The ability to independently produce components also simplifies inventory management. External disinfection spaces of various specifications can be sold individually or as part of a bundle, meeting the needs of different users. This design not only offers users a wider range of choices but also creates opportunities for diversified sales models, providing manufacturers with more flexibility to cater to various market demands.

### BRIEF DESCRIPTION OF THE DRAWINGS

FIG. 1 is a three-dimensional schematic diagram of a portable ozone disinfection device in accordance with an embodiment of the present disclosure;

FIG. 2 is a three-dimensional schematic diagram of a portable ozone disinfection device in accordance with another embodiment of the present disclosure;

FIG. 3 is a schematic diagram illustrating the external disinfection space accommodating respiratory accessories in accordance with various embodiments of the present disclosure;

FIG. 4 is a three-dimensional schematic diagram illustrating the use of a tee connector at the interface to connect multiple external disinfection spaces in accordance with an embodiment of the present disclosure;

FIGS. 5A and 5B are schematic diagrams illustrating different forms of the external disinfection space of an ozone disinfection device in accordance with various embodiments of the present disclosure;

FIGS. 6A, 6B, and 6C are schematic diagrams illustrating different sealing methods used for the external disinfection space in accordance with various embodiments of the present disclosure;

FIG. 7 is a schematic diagram illustrating the portability and storage of an ozone disinfection device in accordance with various embodiments of the present disclosure;

FIG. 8 is a simplified structural overview and sealing schematic diagram of an ozone disinfection device in accordance with various embodiments of the present disclosure;

FIG. 9 is a simplified structural overview and sealing schematic diagram of an integrated ozone disinfection device;

FIG. 10 is a schematic diagram illustrating the flow path of ozone gas in accordance with an embodiment of the present disclosure;

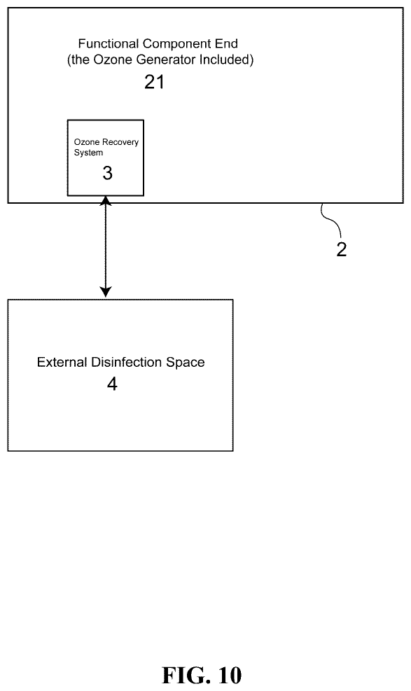

FIG. 11 is a schematic diagram illustrating the flow path of ozone gas in accordance with another implementation of an embodiment of the present disclosure;

FIG. 12 is a three-dimensional schematic diagram of the functional component end of an ozone disinfection device with at least one sensor in accordance with an embodiment of the present disclosure;

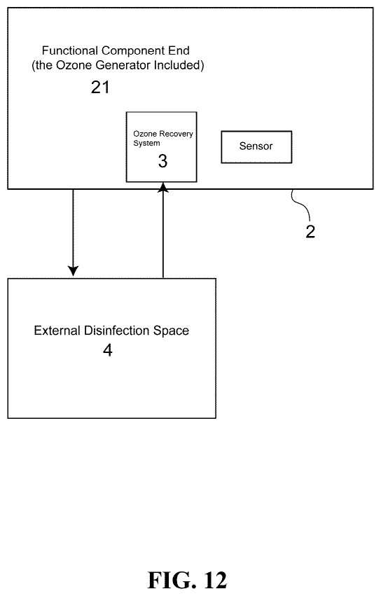

FIG. 13 is a schematic diagram illustrating the flow path of ozone gas in accordance with another embodiment of the present disclosure; and

FIGS. 14A and 14B are schematic diagrams illustrating the ozone recovery system connected separately to the functional component end and the disinfection space in accordance with various embodiments of the present disclosure.

### DETAILED DESCRIPTION

To make the objectives, features, and advantages of this disclosure clearer and easier to understand, the following is a detailed description of specific embodiments of the disclosure, combined with accompanying diagrams. Numerous specific details are discussed below to provide a thorough understanding of the disclosure. However, it is possible to implement the disclosure in many other ways different from those described here, and those skilled in the art can make similar improvements without departing from the spirit of the disclosure; thus, the disclosure is not limited to the specific embodiments disclosed below.

In this disclosure, the functional component end of the ozone disinfection device-that is, the end that generates and transports ozone gas and performs various functional reactions-is physically isolated from an external disinfection space, meaning they are not encased within the same shell. This implementation allows the ozone disinfection device of this disclosure to be smaller in size compared to common disinfection devices on the market, enhancing portability and convenience for the user. The separate external disinfection space can be made from different materials depending on various user needs or manufacturing considerations, greatly increasing the selectivity of the external disinfection space material. At the functional component end, due to the absence of external space constraints, the internal layout can be more organized and compact. Improvements in materials used for the external disinfection space can also enhance the effectiveness and reliability of the ozone disinfection device. Therefore, not only does this disclosure improve performance indicators, but it also provides a more stable, safe, and comfortable user experience.

The following are specific embodiments that illustrate various implementations of the ozone disinfection device 1, in this disclosure.

### Embodiment 1

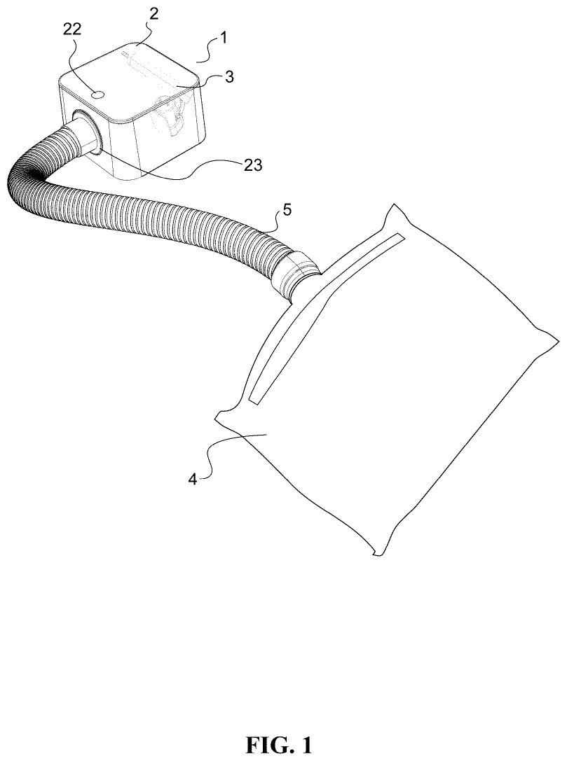

In this disclosure, an ozone disinfection device 1 is provided that is designed for user convenience, enhanced usability, and comfortable wear. Specifically, as detailed in FIGS. 1 to 6, this embodiment of the ozone disinfection device 1 includes: an ozone generation system 21, configured to produce and output ozone gas; an ozone recovery system 3, including at least one pump or fan configured to suction gas; and an external disinfection space 4, fluidly connected to the ozone generation system 21 and configured to accommodate respiratory accessories. The ozone gas is generated and flows from the ozone generation system 21, passes through the external disinfection space 4, and is then recovered by the ozone recovery system 3. Additionally, the ozone disinfection device includes at least one interface 23, configured to fluidly connect to the external disinfection space 4, which is configured to be sealable and gas-impermeable. Furthermore, the ozone disinfection device also includes an ozone operation system 22, configured to control the operation of the ozone disinfection device 1, and the ozone operation system 22 includes at least one switch.

The ozone disinfection device 1 is divided into two main parts: the functional component end, which includes the housing 2, and the external disinfection space 4. The ozone generation system 21 and the ozone recovery system 3 are components of the functional component end and are housed within a separate, individual housing 2. This housing 2 is configured to enclose and support the ozone generation system 21, the ozone recovery system 3, and other functional components, providing stable structural support for the ozone disinfection device 1 during use and transportation, and protecting it from external environmental disturbances. The design of this housing 2 can be varied, tailored to the layout of its internal functional components and the specific functional requirements of the ozone disinfection device 1, ensuring structural stability and enhancing the maintainability of the device. For instance, a modular design of the housing 2 allows for quick disassembly, facilitating maintenance or replacement of internal parts by users or professional technicians, and can also be adjusted in appearance and size specifications according to market demands, improving user experience and market adaptability. High concentrations of ozone can irritate or damage the respiratory tract, eyes, and skin. Prolonged exposure to high concentrations of ozone can also lead to respiratory system damage. Therefore, the housing 2 of the functional component end must be ozone-resistant and sealed, not necessarily formed by the housing 2 itself but possibly by controlling the flow of ozone gas within the pipes inside the housing 2 to prevent it from escaping to the external environment. The material of the housing 2 can vary widely; typically, it may include, but is not limited to, engineering plastics such as acrylonitrile butadiene styrene (ABS), polycarbonate (PC), polyphenylene sulfide (PPS), polypropylene (PP), metals, composite materials, or materials with special coatings or platings. The specific materials for the housing 2 of the ozone disinfection device 1 can be selected based on different usage scenarios, market demands, and other factors.

The functional components enclosed by the housing 2 include at least the ozone generation system 21 and the ozone recovery system 3. The ozone generation system 21 is separated from the external disinfection space 4 and fluidly connected to the ozone recovery system 3. The ozone recovery system 3 is configured to be downstream of the ozone generation system 21, where “downstream” is defined by the path of gas flow, meaning that the ozone recovery system 3 can be located at any position where ozone gas flows out after being generated by the ozone generation system 21. The ozone recovery system 3 includes at least one pump or fan and at least one recovery device. It is configured to connect with the ozone generation system 21 and to recover and filter the ozone gas. The at least one pump or fan is configured to suction gas from the external disinfection space 4 into the at least one recovery device. The at least one recovery device includes at least one layer of recovery material, configured to filter or decompose the disinfected ozone gas into harmless oxygen, preventing ozone from entering the external environment. In cases where the at least one recovery device has multiple layers of recovery material, these layers may possess different filtration functions, such as a physical filtration layer, a chemical adsorption layer, an antimicrobial layer, etc. Generally, the recovery material includes manganese particles for catalyzing the decomposition of ozone gas. In other cases, the at least one recovery device might also use any other form of material capable of catalyzing the decomposition of ozone gas, such as activated carbon, noble metal oxidizers, activated alumina, carbon-based composite materials, or transition metal oxides.

The components at the functional component end are connected via an ozone distribution pipeline, which is used to connect the components and allows ozone gas to circulate among them through the pipeline. The ozone distribution pipeline is made from corrosion-resistant and impermeable materials to prevent ozone leakage during transmission, ensuring the safety of both the device and its users. The layout of the ozone distribution pipeline within the independent housing 2 is flexibly arranged according to the layout of the functional components inside, to achieve efficient gas transport paths in different spatial layouts. In some embodiments, the ozone distribution pipeline can also include check valves or control valves, configured to regulate the flow of air and prevent the backflow of ozone gas, further enhancing the safety and reliability of the ozone disinfection device 1.

Furthermore, the independent housing 2 at the functional component end includes at least one interface 23, which is configured to connect to a hose 5 and fluidly connects to the external disinfection space 4. The hose 5 can be understood as any pipe capable of transporting ozone gas, in any form. The ozone disinfection device 1 also includes at least one gas outlet, located downstream of the external disinfection space 4. This outlet can be provided on any part among the external disinfection space 4, housing 2, or ozone recovery system 3. Typically, the at least one gas outlet is configured to connect with the ozone recovery system 3, ensuring that the emitted ozone is processed into harmless oxygen or low-concentration ozone, meeting environmental protection standards.

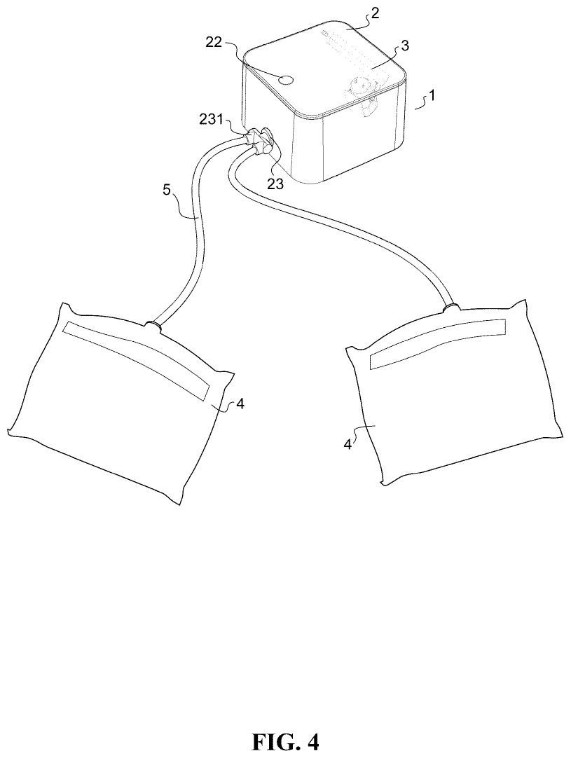

The external disinfection space 4 is a key feature of this disclosure that enhances user experience, serving as a space to receive and accommodate respiratory accessories. It is fluidly connected to the ozone generation system 21 and configured to hold respiratory accessories. As an independent module, the external disinfection space 4 in this disclosure can be individually replaced and maintained, implementing a modular design approach. The external space can have various design forms, offering flexible options to meet different user needs. Users can choose external disinfection spaces 4 of different sizes, functions, and materials, and can also select to use one or multiple external disinfection spaces 4 to match with the functional component end. When the functional component end of the ozone disinfection device 1 is matched with multiple external spaces, the matching can be facilitated through the use of a diverter connection at the at least one interface 23 of the housing 2 or at the end of hose 5 (such as a tee connector 231 or cross connector), thereby distributing ozone gas to multiple external disinfection spaces 4 (as shown in FIG. 4).

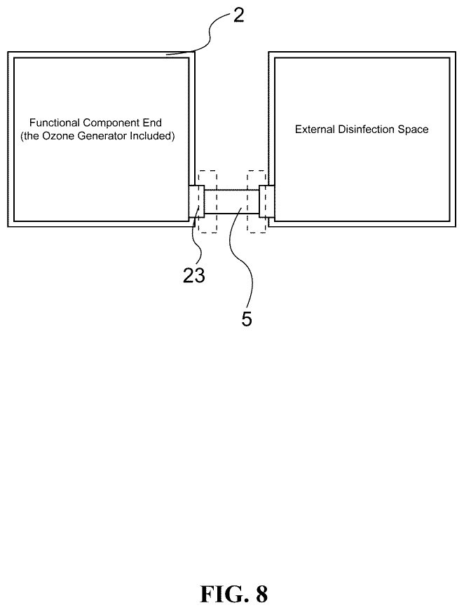

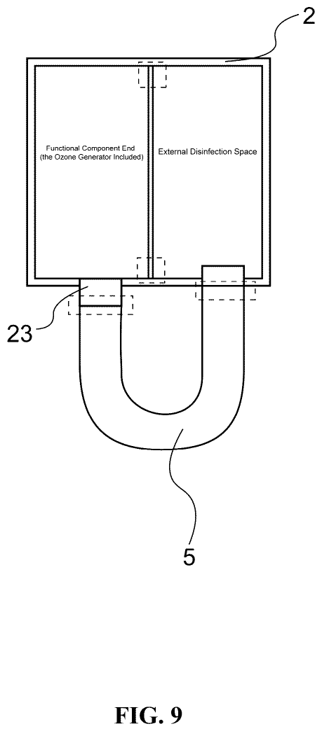

This design of the ozone disinfection device 1 reduces the need for sealing points (as shown in FIGS. 8 and 9). Specifically, by separating the disinfection space from the functional component end in this disclosure, besides the basic seal of the housing 2, the overall ozone disinfection device 1 only requires two sealing points (as indicated by the dashed box in FIG. 8). In contrast, integrated ozone disinfection devices on the current market, where the functional component end and the disinfection space are combined, require at least four sealing points (as indicated by the dashed box in FIG. 9), thus increasing the seals between the disinfection space and the functional component end. Therefore, this disclosure's ozone disinfection device 1 simplifies the assembly and disassembly process and enhances its overall functionality.

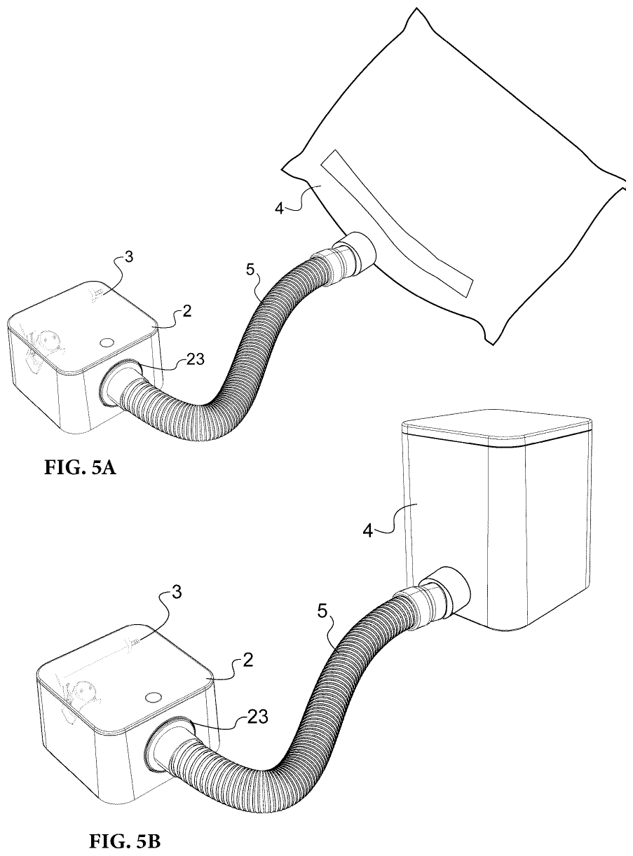

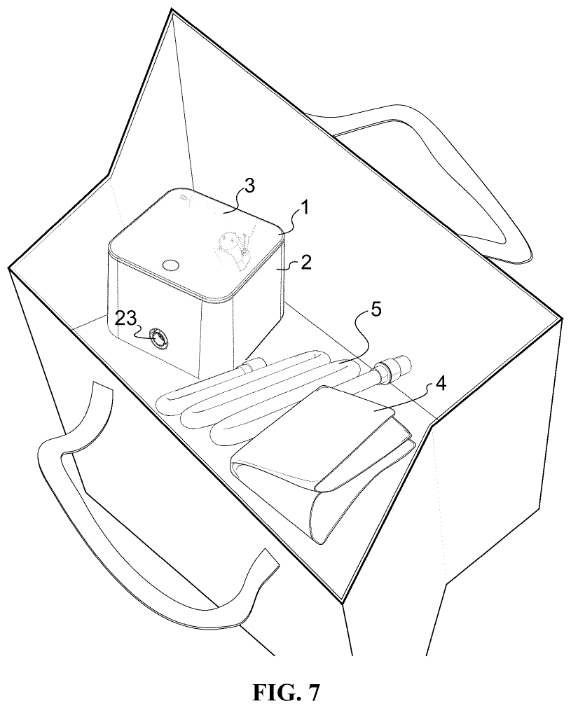

The external disinfection space 4 is made from materials that are sealed and ozone-resistant, ensuring that the ozone effectively functions during the disinfection process without leakage and avoiding the aging or degradation issues that might arise from prolonged exposure to ozone gas. The materials used for the external disinfection space 4 must possess multiple characteristics to ensure efficient, safe, and durable disinfection processes (as shown in FIG. 5, the external disinfection space 4 can also come in various forms). In one form, the external disinfection space 4 is made of a flexible material, specifically, it can be a foldable fabric. This fabric can also be lightweight, making it easy for users to store and carry daily (as shown in FIG. 7). In some cases, this fabric can be repeatedly washed and reused, extending the lifespan of the ozone disinfection device 1. The fabric can be single-layered or multi-layered; in one form, the external disinfection space 4 is a collection of multi-layered materials, where different layers of the fabric can have different effects to collectively form a more suitable external disinfection space 4. In the case where the external disinfection space 4 is of this flexible material type, the materials can include polyester fibers, nylon, polyester, neoprene, and their composites, or coated fabrics. In other implementations, the external disinfection space 4 can also be made from non-fabric rigid materials, such as plastic materials (including but not limited to high-density polyethylene, polypropylene, polycarbonate, and polyvinyl chloride), metal materials (including but not limited to stainless steel, aluminum alloy), silicone and rubber materials, glass, or various composite materials. In this implementation, the external disinfection space 4 can also be either single-layered or multi-layered. When the external disinfection space 4 is of a non-fabric form, portability can be achieved through structural design, such as by making the space foldable, detachable, etc. Using quick assembly or simple fixation mechanisms (such as slides, clips, or magnetic connections) can make the assembly and disassembly of the disinfection space more efficient, further enhancing its portability and ease of operation even when the disinfection space is made of rigid materials. To further meet environmental standards, the external disinfection space 4 could also utilize recyclable or biodegradable materials, such as bioplastics, high-performance composites, or recycled polymers. These materials not only reduce the environmental impact through recycling or natural degradation at the end of the product's life cycle but also decrease reliance on non-renewable resources during production.

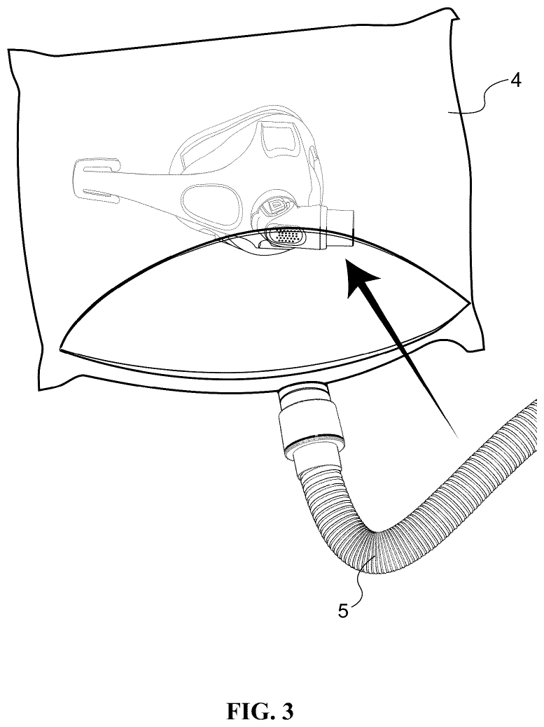

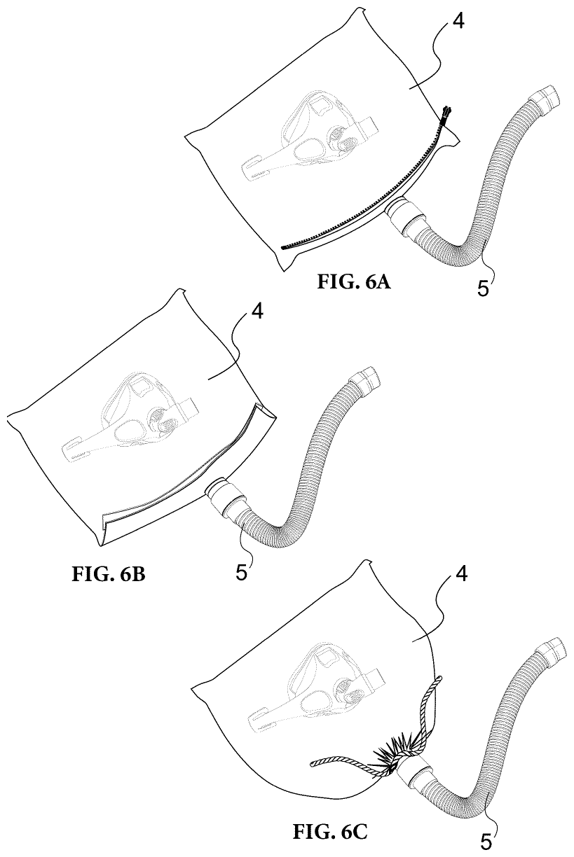

In addition, the external disinfection space 4 includes at least one opening, specifically, the external disinfection space 2 has at least one opening that can switch between open and closed states (as shown in FIG. 3). When the external disinfection space 4 is made of a flexible material, the forms of the at least one opening can include, but are not limited to, zippers, hook and loop fastener, drawstrings, button closures, or combinations of these types (as shown in FIG. 6). When the external disinfection space 4 is made of a rigid material, the forms of the at least one opening can include, but are not limited to, hinge, slide cover, snap fit, twist, bolt, flip cover, spring lid, or threaded types, or combinations of these types. These at least one opening must ensure the airtightness of the external disinfection space 4 when the ozone disinfection device 1 is operating. Therefore, the at least one opening can also include multiple seals to further enhance gas tightness. The at least one interface between the external disinfection space and the hose can be the same as the at least one opening or separate. The form of the at least one interface also varies, including but not limited to elastic deformation connections, structural connections, or adhesive bonds.

In this embodiment, various specifications for the external disinfection space 4 are defined. For example, it is configured to hold a minimum ozone volume of 0.125 liters, enabling it to effectively accommodate the disinfection of various respiratory accessories while maintaining convenience. Additionally, the volume of ozone gas produced by the ozone generation system 21 during operation must be at least one-third of the volume of the external disinfection space 4. This volume is necessary to ensure that the ozone gas can cover the entire external disinfection space 4, thereby achieving effective disinfection results. Furthermore, it is stipulated that the volume of gas suctioned by the at least one pump or fan during operation must be greater than or equal to the volume of the external disinfection space 4. This ensures effective recovery of ozone from the external disinfection space 4 by the ozone recovery system 21, which is a crucial aspect of the efficacy of the ozone disinfection device 1. During the usage of the ozone disinfection device 1, the volume of ozone gas produced by the ozone generation system 21 must be sufficient to maintain an ozone concentration within the external disinfection space 4 that is no lower than the concentration needed to kill standard bacteria found in respiratory equipment and its accessories. This ensures that all bacteria within the respiratory equipment and its accessories are completely eradicated. These accessories include, but are not limited to, masks, nasal masks, hose 5, and frames that are compatible with respirators. Additionally, it is ensured that the volume of gas drawn by one or more fans or pumps in the ozone operation system 22 is not less than the volume of ozone gas produced by the ozone generation system 21. This guarantees that all ozone gas within the external disinfection space 4 can be completely converted into harmless gas by the filtration device, preventing harm to humans or the environment.

The ozone disinfection device can also include an ozone operation system 22, including one or more displays, indicator lights, buttons, and other designs configured to provide users with more straightforward operation and richer functional indications. The user interfaces such as displays and indicator lights can show the progress of the ozone disinfection device 11, including the percentage of total operation time or a countdown of the working time. In other implementations, the operation system 22 of the ozone disinfection device 1 might include additional forms of interaction beyond displays, indicator lights, and buttons, such as voice control. In some implementations, an important feature of the ozone operation system 22 is an alarm function, which is configured to notify the user visually or audibly if there is an anomaly or if the device is not used correctly, thereby preventing the potential for greater hazards. When a malfunction occurs, the alarm system can also interact with other components within the ozone operation system 2 to quickly locate and address the problem area, preventing more severe damage from ongoing operation and also facilitating maintenance personnel in repairing and addressing issues with the device. This alarm system enhances safety for users, optimizes the user experience, and ensures the product can perform effective disinfection safely and reliably. The alarm system can also work in conjunction with the user interface to alert the user.

In this implementation, the disinfection pathway of the ozone disinfection device 1 involves the ozone generation system 21 inside the housing 2 producing ozone gas, which is then transported through the ozone distribution pipeline to the at least one interface 23. From there, the ozone gas is transmitted via the hose 5 connected to the at least one interface 23 to the external disinfection space 4 for disinfecting respiratory equipment or accessories. After disinfection, the ozone gas is recovered through one or more fans or pumps along with a hose 5 back to the ozone recovery system 3 inside the housing 2, and finally expelled from the at least one gas outlet.

In other implementations, the ozone disinfection device 1 also has at least one sensor, including but not limited to ozone sensors, temperature and humidity sensors, pressure sensors, oxygen sensors, flow sensors, volatile organic compound (VOC) sensors, particulate matter sensors, ultraviolet sensors, current sensors, and safety lock sensors, among others. The at least one sensor can be configured to work in conjunction with the alarm system, effectively enhancing the safety and intelligence level of the ozone disinfection device 1. The at least one sensor can also interact with components in the functional component end, such as the ozone generation system 21, to enable intelligent ozone concentration output that users can adjust, providing greater operational flexibility. Additionally, this setup reduces energy or material waste, improves the device's safety and reliability, and fosters greater user trust in the product. The at least one sensor can be installed at any location on the ozone disinfection device, with the at least one sensor at different positions serving distinct functions.

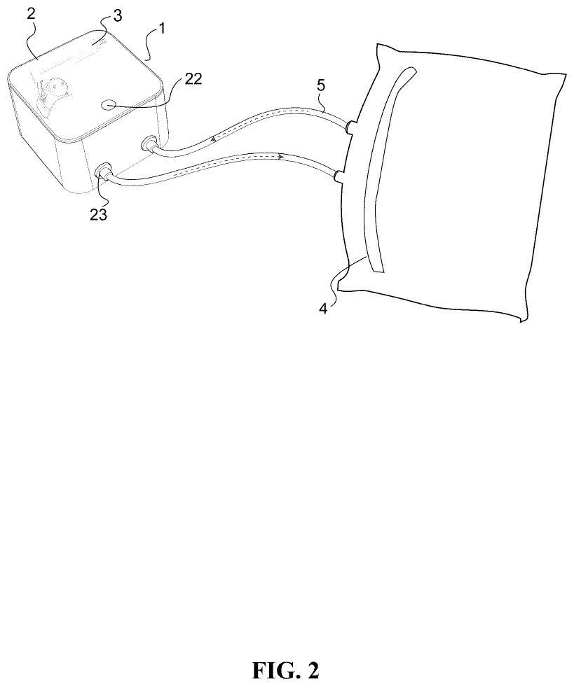

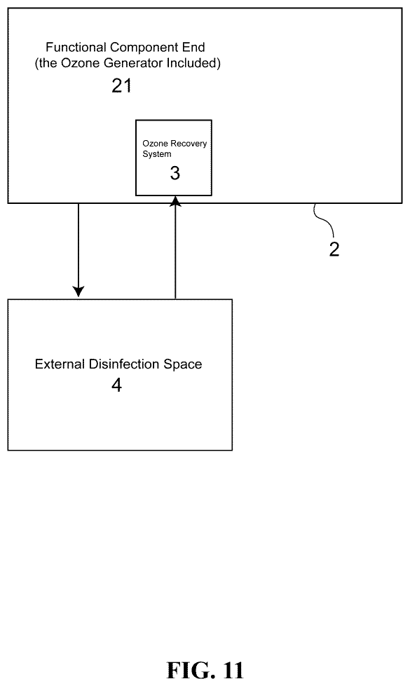

In other implementations, during the ozone recovery process, the hose 5 used for recovering ozone gas is separate from the hose 5 that delivers ozone to the external disinfection space 4 (as shown in FIGS. 2 and 11).

In yet another implementation, the housing 2 does not contain an ozone distribution pipeline. Instead, the housing 2 is made of ozone-resistant material and is configured to be airtight, allowing the ozone gas generated by the ozone generation system 21 within the housing 2 to circulate directly inside the housing.

### Embodiment 2

This embodiment of the ozone disinfection device 1 includes an ozone generation system 21, which is configured to produce and output ozone gas. Additionally, it further includes an ozone recovery system 3, having at least one pump or fan that is configured to suction gas. The ozone disinfection device 1 also incorporates an external disinfection space 4, which is fluidly connectable to the ozone generation system 21. This external disinfection space 4 is specifically configured to accommodate respiratory accessories. Ozone gas is generated by the ozone generation system 21 and flows out to the external disinfection space 4, where it is utilized for disinfection. The ozone gas is then recovered by the ozone recovery system 3. Furthermore, the ozone disinfection device has at least one interface 23, which is configured to fluidly connect to the external disinfection space 4. The external disinfection space 4 is configured to be sealable and gas-impermeable, ensuring effective and safe operation. The device also includes an ozone operation system 22, which is configured to control the operation of the ozone disinfection device. The ozone operation system 22 has at least one switch, providing users with convenient control.

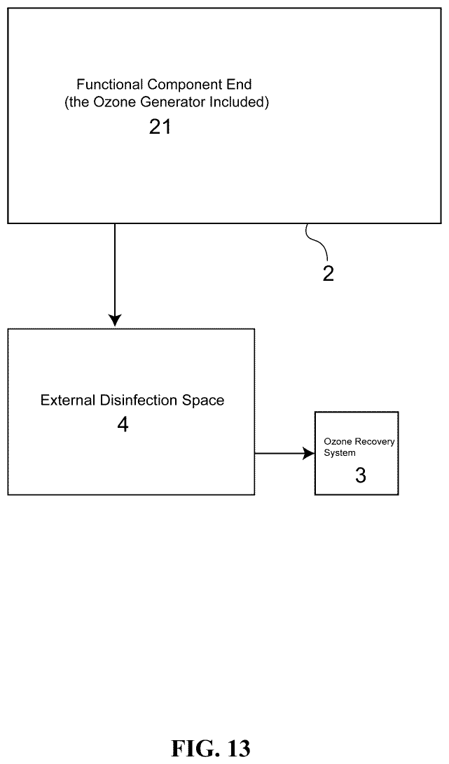

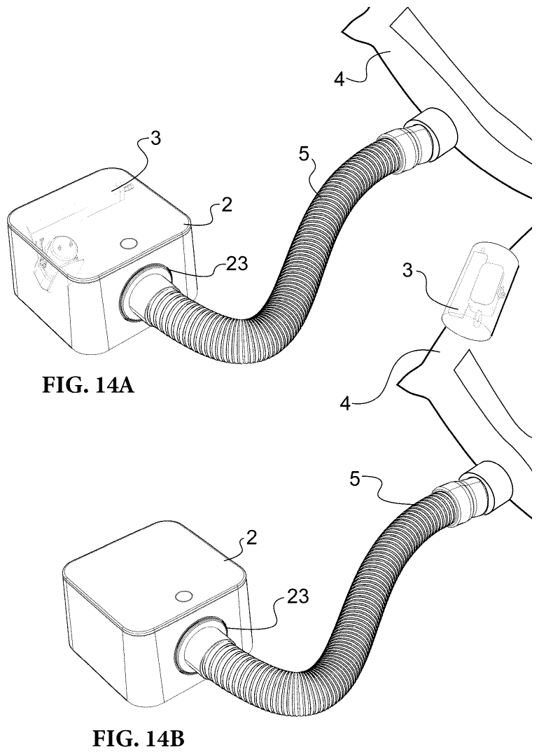

The difference between this embodiment and Embodiment 1 lies in the configuration of the ozone recovery system 3. Here, the ozone recovery system 3 is configured to connect directly to the external disinfection space 4 and perform recovery and filtration (as shown in FIGS. 13 and 14). In this embodiment, the ozone recovery system 3 is set up as a separate component at the end of the external disinfection space 4. This design optimizes the structure of the ozone disinfection device 1 by eliminating the need for ozone gas to return to the functional component end. Instead, the ozone gas is directly recovered and expelled, making the disinfection process more efficient and convenient. In this embodiment, the pathway of the ozone gas is as follows: The ozone generation system 21 within the housing 2 generates ozone gas, which is delivered through the ozone distribution pipeline to the interface 23. The ozone gas is then transferred via the hose 5 connected to the interface 23 to the external disinfection space 4, where it is used to disinfect respiratory accessories. The disinfected gas is then directly expelled from the external disinfection space 4 or the ozone recovery system 3 connected to it, without returning to the housing 2 of the functional component end. This design allows the housing 2 at the functional component end to no longer include the ozone recovery system 3, thereby reducing its size. As a result, the overall structure becomes more compact, significantly improving portability and making the device more suitable for travelers or users needing a temporary disinfection solution. Moreover, the simplified functional component end reduces complexity, lowers the potential failure rate, and enhances the device's reliability and durability, thereby extending its lifespan. This modular design also provides greater convenience for maintenance and replacement, enabling users or technicians to independently inspect, replace, or upgrade the ozone recovery system 3 without disassembling the entire device. This improvement boosts maintenance efficiency and ensures that the device achieves greater flexibility and efficiency during production, use, and maintenance.

Additionally, the technical features described in the above embodiments can be combined as needed to create an ozone disinfection device 1 that incorporates all or part of these features.

The ozone disinfection device 1 of this disclosure offers at least the following beneficial effects:
1) This disclosure provides not only a portable ozone disinfection device 1 for convenient use but also adds a recyclable function compared to existing ozone disinfection devices on the market. This enhancement improves the device's safety, efficiency, and reliability while maintaining its convenience. The portable ozone disinfection device 1 is lightweight and easy to carry, offering significant convenience to users. Unlike integrated ozone disinfection devices currently available, this disclosure is more suited for use in various scenarios, such as during travel or in outdoor environments, thereby enhancing product adaptability and making it less dependent on environmental conditions. This mobility also reduces reliance on specific storage and transportation conditions, lowering costs associated with these processes. Furthermore, compared to existing products on the market, the ozone disinfection device 1 of this disclosure includes a recyclable function that effectively processes ozone gas after use, reducing potential harm to the environment or users. The addition of an ozone recovery system 3 makes this portable ozone disinfection device 1 environmentally beneficial compared to other portable ozone disinfection devices with similar functionalities. It aligns with the requirements of sustainable development. The ozone recovery system 3 ensures that after disinfection, the ozone gas is effectively converted into oxygen, preventing excessive ozone release into the environment. In traditional disinfection processes, ozone gas is often directly released after disinfection, leading to resource waste and increased energy consumption during use. By incorporating the ozone recovery system 3, this device maximizes ozone gas recovery, conserving energy and reducing operational pollution. The ozone recovery system in this disclosure complies with relevant environmental regulations, reducing ozone gas leakage, helping users meet environmental standards, and preventing environmental pollution caused by improper use of the device. In the current era of increased environmental awareness, this eco-friendly design enhances the social responsibility of both users and manufacturers. In summary, the portable ozone disinfection device offers high mobility and adaptability, providing users with an efficient and flexible disinfection experience. It broadens the device's applicability while meeting user demands for a green, efficient, and convenient disinfection device, delivering significant social value.
2) Additionally, the portable ozone disinfection device 1 with recyclable functionality extends the device's lifespan and reduces replacement costs. Ozone gas is highly oxidizing, and prolonged contact with the internal materials of the device can lead to corrosion or aging of key components, resulting in performance degradation. Due to its relatively simple structure and compact design, the portable ozone disinfection device 1 lacks excess complexity. If the internal gas pathways are not fully sealed, there is a high risk of damage to sensors or electronic components, particularly those sensitive to oxidative reactions. This disclosure addresses these challenges by using corrosion-resistant materials for constructing the housing 2 of the ozone disinfection device 1, while simultaneously utilizing the ozone recovery system 3 to minimize ozone gas accumulation inside the device and reduce the storage time of ozone gas within the device. This system prevents gas backflow or dispersal, which is crucial for protecting internal materials and extending the device's lifespan. Moreover, the external disinfection space 4 can be tailored to user requirements by choosing different suitable materials, offering significant flexibility. This broad range of material options not only allows manufacturers to potentially lower material costs and diversify production choices but also enables the external disinfection space to have various characteristics depending on the selected materials. These characteristics might include heat resistance, moisture resistance, UV resistance, corrosion resistance, or lightweight properties, enhancing manufacturing flexibility and, more importantly, the adaptability and practicality of the product. Manufacturers can quickly adjust their designs based on market feedback, ensuring the product consistently aligns with market demands and improves user experience. Furthermore, the customizable materials and optimized design of the external disinfection space 4 can effectively extend the device's lifespan and reduce replacement costs caused by material degradation or functional failure.
3) The portable ozone disinfection device 1 in this disclosure, including an ozone recovery system 3, enhances the user experience when using the device for disinfection. Without the ozone recovery system 3 to automatically reclaim ozone gas during the disinfection process, users would need to manually collect and process the ozone gas or release it directly into the environment. Firstly, high concentrations of ozone gas escaping into the environment could pose risks to users, bystanders, and the surrounding environment. While manually collecting and processing ozone gas is a safer method, it is time-consuming and still carries the risk of gas leakage during handling. By incorporating the ozone recovery system 3 into the portable ozone disinfection device 1, this disclosure simplifies the ozone recovery process, thereby improving the device's convenience and efficiency. In terms of portability, this disclosure uses a portable external disinfection space 4 instead of the larger, heavier integrated external disinfection spaces found in traditional devices. The external disinfection space 4 can feature various design forms and accommodate multiple configurations without being constrained by the functional components or housing 2 of the ozone disinfection device 1. For example, in one implementation, the external disinfection space 4 is made of flexible materials such as fabric, allowing it to be folded when not in use. This design increases the capacity of the external disinfection space 4 for accommodating respiratory-related equipment or respiratory accessories during use while reducing the overall size of the ozone disinfection device 1 when not in use, thereby significantly improving portability. This flexibility not only makes the ozone disinfection device 1 easier to carry daily but also enhances its practicality by meeting diverse disinfection needs. It allows users to disinfect larger or multiple pieces of respiratory equipment and accessories, thereby improving disinfection efficiency.
4) The external disinfection space 4 makes the ozone disinfection device more compact, structurally optimized, and cost-efficient. Furthermore, when combined with the advantages of the ozone recovery system, the overall cost of the ozone disinfection device 1 is further reduced. This disclosure centralizes the design of the functional components while independently designing the external disinfection space 4, which is fluidly connectable to the functional components. This approach not only reduces the need for complex internal wiring and component space but also lowers the requirements for internal space and disinfection space integration. As a result, the size of the housing 2 and the amount of material required are significantly reduced. These optimizations make storage and transportation of the ozone disinfection device 1 more convenient and cost-effective. Cost savings are further reflected in the independent and integrated design of the functional components, which simplifies maintenance and replacement, reducing the complexity of disassembly and reassembly. Since the external disinfection space 4 is a separate unit, the sealing between the two spaces does not require intricate sealing structure designs or multiple testing phases. In production, this disclosure simplifies assembly, further reducing labor and time costs. Moreover, the portable ozone disinfection device 1 offers more user-friendly operation compared to integrated ozone disinfection devices. Its functional simplicity makes it easier for users to understand and operate, reducing the learning curve. This is particularly valuable for users who need frequent disinfection or for elderly users, as it provides a more accessible and convenient experience. In one embodiment, the ozone recovery system 3 is configured to be replaceable as a standalone component. This independent configuration further enhances the usability of the ozone disinfection device 1 by reducing the frequency and complexity of user maintenance. Users only need to periodically check the usability of the individual components of the ozone recovery system 3 and replace it independently when necessary. This modular approach minimizes maintenance efforts while ensuring long-term functionality.
5) The ozone disinfection device 1 in this disclosure provides users with a modular usage option, designed with a full consideration of diverse user needs and future upgrade potential. The external disinfection space 4 allows for flexibility, enabling users to add, reduce, or replace the external disinfection space 4 based on their needs without affecting functionality. For users requiring larger external disinfection spaces 4, they can purchase additional or larger external disinfection spaces 4 individually, or increase the number of external disinfection spaces 4, thereby catering to a wide range of user requirements. This modular design also significantly facilitates product iteration and upgrades. For developers, upgrading the product becomes easier, as improvements to the functional component end only require adjustments and upgrades to core module components without altering the structure or internal circuitry. Moreover, such changes do not necessitate adjustments to the external disinfection space 4. Similarly, if the external disinfection space 4 needs an upgrade, only its materials, size, or other features need to be redeveloped and tested, with no impact on the functional component end's structure or components. This modular approach to research and development shortens the development cycle and reduces overall development costs. Additionally, for manufacturers, the functional component end of the portable ozone disinfection device 1 and the external disinfection space 4 can be produced independently, without the need for additional assembly. This simplifies the production process, reduces assembly complexity, and enhances production efficiency. The ability to independently produce components also simplifies inventory management. External disinfection spaces of various specifications can be sold individually or as part of a bundle, meeting the needs of different users. This design not only offers users a wider range of choices but also creates opportunities for diversified sales models, providing manufacturers with more flexibility to cater to various market demands.

The technical features of the embodiments described above can be combined in any manner. For the sake of brevity, not all possible combinations of the technical features in the above embodiments have been described. However, as long as there is no conflict between these combinations, they should be considered within the scope of this specification.

The embodiments described above represent only a few implementations of this disclosure. While the descriptions are specific and detailed, they should not be understood as limitations to the scope of the patent. It should be noted that for those skilled in the art, various modifications and improvements can be made without departing from the concept of the disclosure, and these are all within the scope of the disclosure. Therefore, the scope of protection of this patent should be defined by the appended claims.

It must be noted that as used herein and in the appended claims, the singular forms “a”, “an”, and “the” include their plural equivalents, unless the context clearly dictates otherwise.

1. A portable ozone disinfection device configured to disinfect and sterilize respiratory-related equipment or respiratory accessories, consisting of:
a housing including an ozone generation system, configured to produce and output ozone gas;
an ozone recovery system, comprising at least one pump or fan, the at least one pump or fan being configured to suction gas; and
an external disinfection space external and separate from the housing, configured to be fluidly connectable to the ozone generation system and to accommodate the respiratory accessories;
wherein, during operation, the external disinfection space is in a closed state such that the ozone gas is generated and flows out in a single path from the ozone generation system, and only the ozone gas is able to pass through the external disinfection space, and is subsequently recovered by the ozone recovery system, wherein the ozone recovery system is a separate component from the housing and configured in a way such that the ozone gas from the external disinfection space is not recovered to the ozone generation system;
wherein the ozone disinfection device further comprises at least one interface, the at least one interface being configured to fluidly connect to the external disinfection space;
wherein, during operation, a volume of the ozone gas generated by the ozone generation system is proportional to at least one-third of a volume of the external disinfection space; and
wherein the external disinfection space is configured to be sealable and gas-impermeable and is configured to accommodate a minimum ozone volume of 0.125 liters.

2. The ozone disinfection device according to claim 1, wherein the ozone disinfection device further comprises an ozone operation system configured to control an operation of the ozone disinfection device, and wherein the ozone operation system includes at least one switch.

3. The ozone disinfection device according to claim 1, wherein the ozone recovery system further comprises at least one recovery device.

4. The ozone disinfection device according to claim 1, wherein the ozone recovery system is configured to connect to the external disinfection space and perform recovery and filtration.

5. The ozone disinfection device according to claim 1, wherein the ozone recovery system is configured to connect with the ozone generation system and perform recovery and filtration.

6. A portable ozone disinfection device configured to disinfect and sterilize respiratory-related equipment or respiratory accessories, consisting of:
a housing comprising an ozone generation system, configured to produce and output ozone gas;
an ozone recovery system, comprising at least one pump or fan, the at least one pump or fan being configured to suction gas, wherein the ozone recovery system further comprises at least one recovery device; and
an external disinfection space external and separate from the housing, configured to be fluidly connectable to the ozone generation system and configured to be sealable and gas-impermeable, and to accommodate the respiratory accessories;
wherein the ozone disinfection device further comprises at least one interface, the at least one interface being configured to fluidly connect to the external disinfection space;
wherein, during operation, a volume of the ozone gas generated by the ozone generation system is proportional to at least one-third of a volume of the external disinfection space;
wherein the ozone recovery system is a separate component from the housing and configured in a way such that the ozone gas from the external disinfection space is not recovered to the ozone generation system; and
wherein the external disinfection space includes at least one opening configured to switch between an open state and a closed state.

7. The ozone disinfection device according to claim 6, wherein the ozone disinfection device further comprises an ozone operation system configured to control an operation of the ozone disinfection device, and wherein the ozone operation system includes at least one switch.

8. The ozone disinfection device according to claim 6, wherein the external disinfection space includes a flexible material.

9. The ozone disinfection device according to claim 6, wherein the external disinfection space includes a rigid material.

10. The ozone disinfection device according to claim 6, wherein the ozone generation system is configured to be separated from the external disinfection space.

11. (canceled)

12. A portable ozone disinfection device configured to disinfect and sterilize respiratory-related equipment or respiratory accessories, consisting of:
a housing comprising an ozone generation system, configured to produce and output ozone gas;
an ozone recovery system, comprising at least one pump or fan, the at least one pump or fan being configured to suction gas; and
an external disinfection space external and separate from the housing, configured to be fluidly connectable to the ozone generation system and configured to be sealable and gas-impermeable, and to accommodate the respiratory accessories;
wherein, during operation, the external disinfection space is in a closed state such that the ozone gas is generated and flows out in a single path from the ozone generation system, and only the ozone gas is able to pass through the external disinfection space, and is subsequently recovered by the ozone recovery system, wherein the ozone recovery system is a separate component from the housing and configured in a way such that the ozone gas from the external disinfection space is not recovered to the ozone generation system;
wherein the ozone disinfection device further comprises at least one interface, the at least one interface being configured to fluidly connect to the external disinfection space;
wherein a volume of the gas suctioned by the at least one pump or fan during operation is greater than or equal to a volume of the external disinfection space; and
wherein the external disinfection space includes at least one opening configured to switch between an open state and a closed state.

13. The ozone disinfection device according to claim 12, wherein the ozone disinfection device further comprises an ozone operation system configured to control an operation of the ozone disinfection device, and wherein the ozone operation system includes at least one switch.

14. The ozone disinfection device according to claim 12, wherein the ozone recovery system is configured to be downstream of the ozone generation system.

15. The ozone disinfection device according to claim 12, wherein the ozone recovery system further comprises at least one recovery device.

16. The ozone disinfection device according to claim 12, wherein the ozone disinfection device further comprises at least one gas outlet, and wherein the at least one gas outlet is provided downstream of the external disinfection space.

17. A portable ozone disinfection device configured to disinfect and sterilize respiratory-related equipment or respiratory accessories, consisting of
a housing comprising an ozone generation system, configured to produce and output ozone gas;
an ozone recovery system, comprising at least one pump or fan, the at least one pump or fan being configured to suction gas; and
an external disinfection space external and separate from the housing, configured to be fluidly connectable to the ozone generation system and configured to be sealable and gas-impermeable, and to accommodate the respiratory accessories;
wherein, during operation, the external disinfection space is in a closed state such that the ozone gas is generated and flows out in a single path from the ozone generation system, and only the ozone gas is able to pass through the external disinfection space, and is subsequently recovered by the ozone recovery system, wherein the ozone recovery system is a separate component from the housing and configured in a way such that the ozone gas from the external disinfection space is not recovered to the ozone generation system;
wherein the ozone disinfection device further comprises at least one interface, the at least one interface being configured to fluidly connect to the external disinfection space;
wherein, during operation, a volume of the ozone gas generated by the ozone generation system is at least one-third of a volume of the external disinfection space;
wherein the ozone disinfection device further comprises an ozone operation system configured to control an operation of the ozone disinfection device, and wherein the ozone operation system includes at least one switch; and
wherein the external disinfection space includes at least one opening configured to switch between an open state and a closed state.

18. The ozone disinfection device according to claim 17, wherein the ozone recovery system further comprises at least one recovery device.

19. The ozone disinfection device according to claim 17, wherein the external disinfection space includes a collection of multi-layered materials.

20. The ozone disinfection device according to claim 17, wherein the external disinfection space includes a single-layer material.

21. The ozone disinfection device according to claim 17, wherein the ozone disinfection device further comprises at least one gas outlet, wherein the at least one gas outlet is provided downstream of the external disinfection space.
